(19) United States
(12) Patent Application Publication
(10) Pub. No.: US 2017/0028145 A1
(43) Pub. Date: Feb. 2, 2017

(54) METHOD AND SYSTEM FOR CONTROLLING PATIENT VENTILATION

(71) Applicant: GENERAL ELECTRIC COMPANY, Schenectady, NY (US)

(72) Inventor: Russell James Kuzelka, McFarland, WI (US)

(21) Appl. No.: 14/813,837

(22) Filed: Jul. 30, 2015

(51) Int. Cl. A61M 13/00; A61M 16/00

(52) U.S. Cl. CPC A61M 13/003; A61M 16/0069; A61M 16/0051; A61M 2205/3303; A61M 2016/0027; A61M 2230/432; A61M 2230/205; A61M 2230/04; A61M 2205/3331; A61M 2205/502

(57) ABSTRACT

A method of ventilating a patient is provided. The method comprises measuring a patient physiological parameter while the patient is in a first position and recording the measured patient physiological parameter as a baseline. The method further comprises tilting the patient from the first position to a second position and measuring the patient physiological parameter when the patient is in the second position. The method further comprises performing an action based on a change in the measured physiological parameter from the baseline when the patient is in the second position. The action is at least one of modifying a ventilation parameter, generating an alert, and providing a recommendation.

### BACKGROUND OF THE INVENTION

The subject matter disclosed herein relates a method and system for ventilation of a patient, and more specifically to ventilatory compensation during laparoscopic insufflation and Trendelenburg patient positioning.

Laparoscopic surgery is a modern surgical technique in which surgical operations to the abdomen are performed through small incisions rather than large incisions used during open laparotomy. During laparoscopic procedures, the abdomen is insufflated with a gas, which is often carbon dioxide. The pneumoperitoneum created by insufflation distends the abdomen wall above the internal organs like a dome to create a working and viewing space. Carbon dioxide (CO2) is used because it is a gas common to the human body, can be absorbed by the tissues, is removed through ventilation and is non-flammable.

In certain surgical procedures pneumoperitoneum may be combined with positioning the patient in Trendelenburg. Trendelenburg is when the body is laid flat on the back in the supine position and tilted from horizontal so the feet are higher than the head. Mild Trendelenburg is between 1 and 30 degrees from horizontal and steep Trendelenburg is between 30 and 45 degrees from horizontal. Reverse Trendelenburg is defined as the body laid flat on the back in the supine position and tilted from horizontal so the head is higher than the feet.

Pneumoperitoneum, and the resultant CO2 absorption, combined with Trendelenburg introduces patho-physiologic changes that complicate anesthetic management for patients undergoing laparoscopic procedures. The increase in intra-abdominal pressure during pneumoperitoneum causes cranial shift of the end-expiratory position of the diaphragm, further reducing end-expiratory lung volume (EELV) and predisposes the patient to airway closure and collapse of dependent lung regions. The upward displacement of the patient's diaphragm can result in reduced lung compliance, reduced Functional Residual Capacity (FRC), an increase in airway resistance, and a ventilation perfusion mismatch with resultant hypercarbia and hypoxemia. The cephalad displacement of the patients mediastinum (toward the head) during Trendelenburg is also capable of causing endobronchial intubation resulting in inadequate delivery of gaseous anesthesia, hypoventilation, hypoxia and cyanosis. The diaphragm shift due to pneumoperitoneum, is further exacerbated in Trendelenburg as the intra-abdominal organs are moved cephalad due to gravity. Obese patients, and those with underlying respiratory disease processes, have a greater risk of atelectasis than non-obese patients.

Currently, a clinician must monitor multiple disparate devices to obtain the information needed to effectively manage a ventilated and insufflated patient in Trendelenburg. These devices may include an insufflation device, a patient monitor, a ventilator, a patient table, and the patient's medical record.

Therefore, it is desirable to have a consolidation of the pertinent physiological, ventilation and procedural data to free the clinician from having to balance multiple disparate sources of data that are critical to patient management during laparoscopic procedures. It is further desirable to have a system and method to provide an automated mode to control ventilator settings as well as to provide clinical decision support to enable the clinician to more efficiently manage a ventilated patient during laparoscopic procedures.

The above-mentioned shortcomings, disadvantages and problems are addressed herein which will be understood by reading and understanding the following specification.

### BRIEF DESCRIPTION OF THE INVENTION

In an embodiment, a method of ventilating a patient is provided. The method comprises measuring a patient physiological parameter while the patient is in a first position and recording the measured patient physiological parameter as a baseline. The method further comprises tilting the patient from the first position to a second position and measuring the patient physiological parameter when the patient is in the second position. The method further comprises performing an action based on a change in the measured physiological parameter from the baseline when the patient is in the second position. The action is at least one of modifying a ventilation parameter, generating an alert, and providing a recommendation.

In another embodiment, a method of ventilating a patient is provided. The method comprises ventilating the patient at a first ventilation setting and measuring at least one physiological parameter from the patient at a horizontal position and storing the measured parameter as a baseline. The method further comprises tilting the patient from the horizontal position to a Trendelenburg position and measuring a patient physiological parameter when the patient is in the Trendelenburg position. The method further comprises comparing the measured patient physiological parameter to the baseline and performing an action based on the comparison of the measured physiological parameter to the baseline, wherein the action is at least one of modifying the first ventilation setting to a second ventilation setting, generating an alert, and providing a recommendation.

In another embodiment, a system for monitoring a patient ventilated in the Trendelenburg position is provided. The system comprises a processor configured to receive a physiological parameter, a ventilation parameter and a procedural parameter. The controller is further configured to generate an indicator representative of the patient's response to the Trendelenburg position. The system further comprises a display operatively connected to the processor and configured to present a pneumoperitoneum dashboard comprising the physiological parameter, the ventilation parameter, the procedural parameter, and the indicator.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of a ventilation system in accordance with an embodiment of the disclosure;

FIG. 2 illustrates a display presenting a pneumoperitoneum dashboard in accordance with an embodiment of the disclosure;

FIG. 3 is a flow chart illustrating a method of ventilating a patient in accordance with an exemplary embodiment of the disclosure; and

FIG. 4 is a flow chart illustrating a method of ventilating a patient in accordance with an exemplary embodiment of the disclosure.

### DETAILED DESCRIPTION OF THE INVENTION

In the present description, certain terms have been used for brevity, clearness and understanding. No unnecessary limitations are to be applied therefrom beyond the requirement of the prior art because such terms are used for descriptive purposes only and are intended to be broadly construed. The different systems and methods described herein may be used alone or in combination with other systems and methods. Various equivalents, alternatives and modifications are possible within the scope of the appended claims. Each limitation in the appended claims is intended to invoke interpretation under 35 U.S.C. §112, sixth paragraph, only if the terms “means for” or “step for” are explicitly recited in the respective limitation.

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

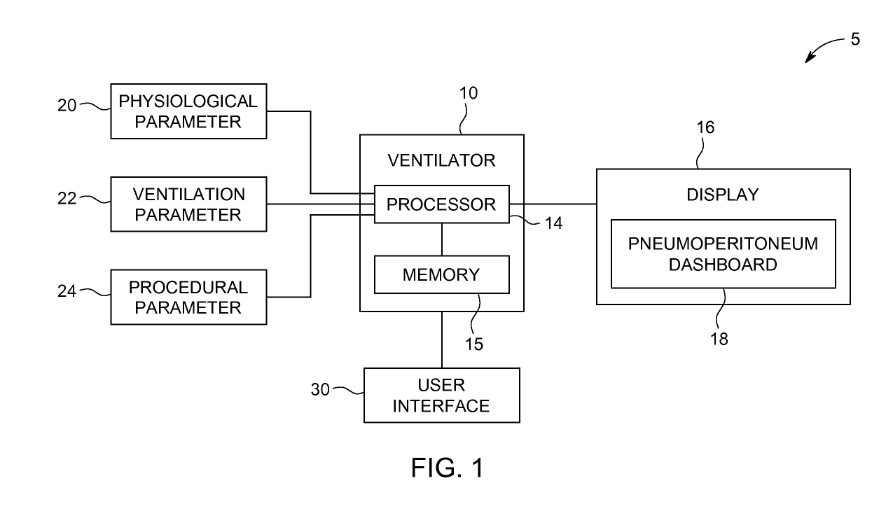

FIG. 1 is a schematic diagram of a ventilation system 5 in accordance with an embodiment of the disclosure. The ventilation system 5 comprises a ventilator 10 operatively connected to a display 16 and a user interface 30. It should be appreciated that, while in FIG. 1, the display 16 and user interface 30 are shown as separate from the ventilator 10, the display 16 and/or the user interface 30 may be integrated into the ventilator 10 while within the scope of the present disclosure.

The ventilator 10 is configured to deliver ventilation gas to a patient (not shown) through an inspiratory limb (not shown) and remove exhaled gases from the patient though an expiratory limb (not shown). The ventilator 10 may be an invasive delivery device, configured to deliver and remove ventilation gases via a tracheal tube, or a non-invasive device configured to deliver and remove ventilation gases via a gas mask. It should be appreciated that various different types of ventilators, including operating components, could be utilized while within the scope of the present disclosure.

The ventilator 10 comprises a processor 14 operatively connected to a memory 15. Memory 15 may be a non-transitory computer-readable medium and may be configured to store computer executable code to be processed by the processor 14 in order to effectuate the ventilator 10. The memory 15 may also be configured to store data received by the processor 14.

The processor 14 is configured to receive data. In one embodiment, data may be input by a user via the user interface 30 that is operationally connected to the processor 14. User interface 30 may include a mouse, a keyboard, a voice input device, a touch input device for receiving a gesture from a user, a motion input device for detecting non-touch gestures and other motions by a user, and other comparable input devices and associated processing elements capable of receiving user input from a user, or a combination thereof. In another embodiment, the processor 14 may be operatively connected to another device, such as a patient parameter sensor, a patient monitor, an insufflation device, a patient table, or a combination thereof, that provides data to the processor 14.

The processor 14 is configured to receive a patient physiological parameter 20. The patient physiological parameter 20 may be intra-abdominal pressure (IAP), peak inspiratory pressure (PIP), end-tidal CO2 (ETCO2), blood oxygen saturation (SPO2), and blood pressure (BP) (including Systolic, Diastolic, Mean), Airway Plateau Pressure (APP), Intracranial Pressure (ICP) or any combination thereof.

The processor 14 is also configured to receive a ventilation parameter 22. The ventilation parameter 22 may comprise a measured tidal volume (TV), respiratory rate (RR), inspired oxygen fraction (Fi02), a ratio of inspiration duration to expiration duration (I:E ratio), positive end expiratory pressure (PEEP), or any combination thereof.

The processor 14 is further configured to receive a procedural parameter 24. The procedural parameter 24 may comprise insufflation gas type, table angle with respect to horizontal, operative time, or any combination thereof. The operative time may be the length of time the patient is in a position, such as Trendelenburg.

In response to receiving a patient physiological parameter 20, a ventilation parameter 22 and a table parameter 24, the processor 14 may be configured to generate an indicator representative of the patient's response to the Trendelenburg position. In one embodiment, the indicator may be a numeric value. For example, the numeric value may be a number between 1 and 10, wherein 1 represents a patient responding positively to Trendelenburg and 10 represents a patient in distress. In another embodiment, the indicator is color. For example, colors along a green/yellow/red spectrum may be used to qualify a patient's response to Trendelenburg, where green represents a patient responding positively to Trendelenburg and red represents a patient in distress. In yet another embodiment, the indicator may be a combination of a numeric value and a color. It should be appreciated however, that other embodiments of the indicator may be envisioned.

The processor 14 is configured to perform an action based on processing the received physiological parameter 20, the ventilation parameter 22 and the procedural parameter 24. The action may comprise at least one of generating an alert, providing a recommendation, and modifying a ventilation setting.

The alert may be indicative of a physiological parameter value being outside a pre-determined threshold value. The alert may be audible, comprising a tone or pre-recorded message. The alert may comprise a visual indication on the display 16. In one embodiment, the visual indication may be a series of words alerting to a possible complication. In another embodiment, the visual indication may be a flashing or highlighting of a patient parameter 20, a ventilation parameter 22 or a procedural parameter 24. In yet another embodiment, color may be used to draw a clinician's attention to a particular patient parameter 20, ventilation parameter 22, or procedural parameter 24. For example, as the patient's IAP increases, the parameter box, text, or value, or any combination thereof, will change from green to yellow to red. It should be appreciated that various other types or embodiments of visual alerts may be envisioned within the scope of the present disclosure.

Providing a recommendation may comprise providing clinical decision support to guide the clinician in managing the patient ventilation. In one embodiment, a change in ventilation setting may be recommended. For example, it may be recommended to decrease tidal volume (TV), increase respiratory rate (RR), manage PIP to be less than 35 cm H20, increase positive end-expiratory pressure (PEEP), perform a recruitment maneuver, reduce the I:E ratio to 1:1 or initiate an inverse I:E ratio whereby the inspiratory time is longer than the expiratory time, increase FiO2, or any combination thereof. In another embodiment, a change in a procedural parameter may be recommended. For example, it may be recommended to change the patient position, such as reducing the table tilt from a steep Trendelenburg to a mild Trendelenburg. It should be appreciated, however, that other recommendations may be envisioned within the scope of the present disclosure.

Modifying a ventilation setting may comprise the processor 14 automatically adjusting at least one of tidal volume, respiratory rate, inspired oxygen fraction, and I:E ratio, or any combination thereof. It should be appreciated that modifications to other ventilation settings may be envisioned within the scope of the present disclosure. For example, in response to the patient being positioned in Trendelenburg, the processor 14 may decrease tidal volume, increase respiratory rate, manage PIP to be less than 35 cm H2O, increase Positive end-expiratory pressure (PEEP), perform a recruitment maneuver, reduce the I:E ratio to 1:1 or initiate an inverse I:E ratio whereby the inspiratory time is longer than the expiratory time, increase FiO2, or any combination thereof.

The processor 14 may be operatively connected to a graphical display 16. The display 16 may be integrated into the ventilator 10, or may be separate from the ventilator 10 as shown in FIG. 1. The display 16 is configured to convey information relating to the ventilator 10, such as current ventilation parameters 22 or ventilation settings, and/or the ventilated patient, such as patient physiological parameters 20. In one embodiment, the display 16 may comprise a graphical user interface (not shown) configured to receive user input.

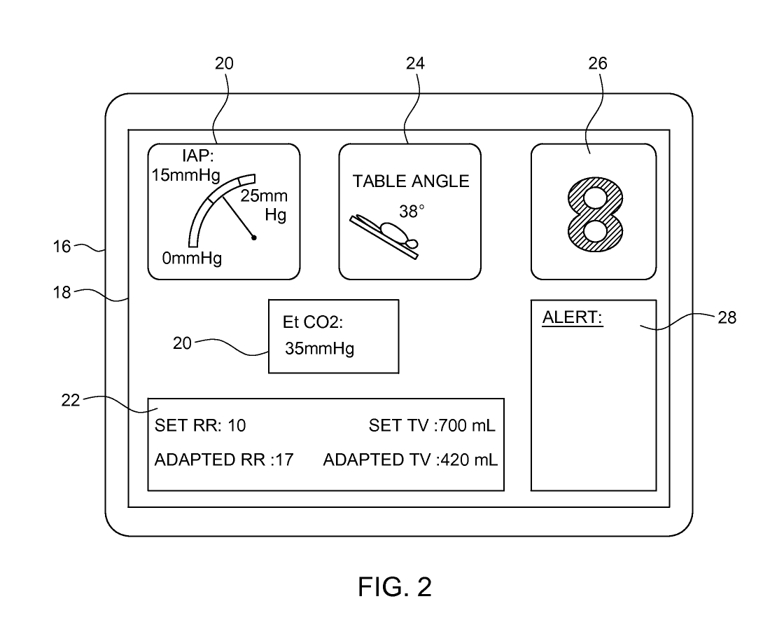

Referring to FIG. 2, the display 16 may be configured to convey a pneumoperitoneum dashboard 18. The dashboard 18 may present at least a physiological parameter 20, a ventilation parameter 22 and a procedural parameter 24, but may present a combination thereof. Additionally, the dashboard may present a plurality of physiological parameters 20, a plurality of ventilation parameters 22, a plurality of procedural parameters 24, or a combination thereof.

The physiological parameter 20 may comprise IAP, PIP, ETCO2, SPO2, BP, APP, and ICP, or any combination thereof. The ventilation parameter 22 may comprise tidal volume, respiratory rate, inspired oxygen fraction, I:E ratio, PEEP, or any combination thereof. In the embodiment depicted in FIG. 2, the ventilation parameter 22 comprises current ventilation parameters Set RR and Set TV. The dashboard 18 may also be configured to convey a ventilation setting, such as Adapted RR or Adapted TV, to which the ventilator is driving towards. Procedural parameters 24 may comprise insufflation gas type, table angle, operative time, or any combination thereof.

In the embodiment depicted in FIG. 2, a combination of words, numeric values, graphs, and symbols are used to depict physiological parameter 20, ventilation parameter 22 and procedural parameter 24. It should be appreciated, however, that other embodiments of these parameter depictions may be envisioned within the scope of the present disclosure.

The pneumoperitoneum dashboard 18 may be further configured to present the indicator 26 generated by the processor 14. As described above with reference to FIG. 1, the indicator is representative of the patient's response to the Trendelenburg position. In one embodiment, the indicator may be a numeric value. For example, the numeric value may be a number between 1 and 10, wherein 1 represents a patient responding positively to Trendelenburg and 10 represents a patient in distress. In another embodiment, the indicator is color. For example, colors along a green/yellow/red spectrum may be used to qualify a patient's response to Trendelenburg, where green represents a patient responding positively to Trendelenburg and red represents a patient in distress. In yet another embodiment, the indicator may be a combination of a numeric value and a color. It should be appreciated however, that other embodiments of the indicator may be envisioned.

The pneumoperitoneum dashboard 18 may also be configured to present clinical decision support 28. In the embodiment depicted in FIG. 2, clinical decision support 28 is conveying an alert. However, it should be appreciated that other forms of clinical decision support may be presented. For example, the clinical decision support 28 may comprise a recommendation for modifying a ventilation setting. In another example, the clinical decision support 28 may comprise a status reflecting a modification of a ventilation setting made automatically by the processor 14 in response to the physiological parameter 20, the ventilation parameter 22 and the procedural parameter 24.

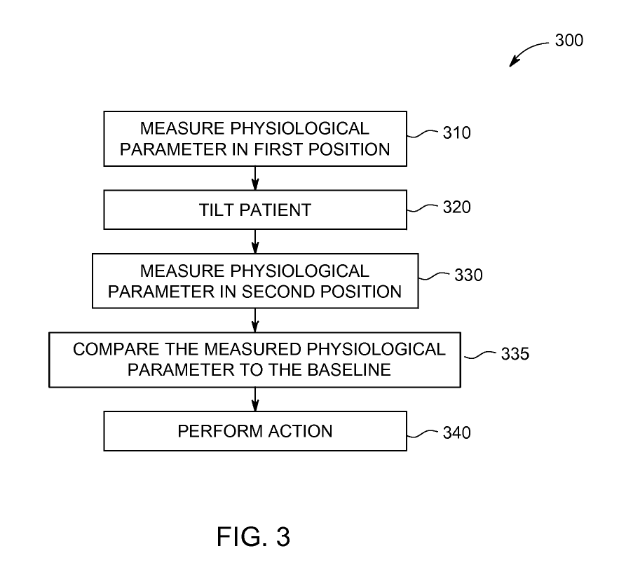

FIG. 3 is a flow chart in accordance with an embodiment of a method 300 of ventilating a patient. At step 310, at least one patient physiological parameter is measured while the patient is in a first position. In one embodiment, the first position is the patient supine on its back horizontal to the ground. In another embodiment, the first position may be a mild Trendelenburg position, where the patient is titled from horizontal such that the patient's legs are higher than the patients head, and the patient is between 1 and 30 degrees from horizontal.

The patient physical parameter may be intra-abdominal pressure, peak inspiratory pressure, end-tidal CO2, blood oxygen saturation, blood pressure, airway plateau pressure, intracranial pressure, or a combination thereof. The measured patient physiological parameter or parameters are recorded as a baseline for the parameter and stored in the memory of the ventilator. The baseline represents a pre-procedure value of the parameter prior to beginning of the tilting of the patient.

At step 320, the patient is tilted from the first position to a second position different, wherein the second position is different from the first position. In one embodiment, the patient is tilted from a horizontal position to either one of a mild Trendelenburg or a steep Trendelenburg. In another embodiment, the patient is tilted from one position within the mild Trendelenburg range to another position within the mild Trendelenburg range. In yet another embodiment, the patient is tilted from a mild Trendelenburg to a steep Trendelenburg.

At step 330, when the patient is in the second position, the patient physiological parameter is measured.

At step 335, the measured patient physiological parameter or parameters is compared to the baseline value measured and recorded before the patient was tilted to a more severe Trendelenburg position.

At step 340, an action is performed. The action is based on the comparison between the baseline and the subsequently measured physiological parameter when the patient is in the second position. The actions may be at least one of modifying a ventilation parameter, generating an alert, and providing a recommendation, or may be any combination thereof.

Modifying a ventilation parameter may comprise modifying, by the processor, at least one of tidal volume, respiratory rate, inspired oxygen fraction, I:E ratio, PEEP, or a combination thereof. It may also comprise the processor 14 decreasing tidal volume, increasing respiratory rate, managing PIP to be less than 35 cm H2O, increasing PEEP, performing a recruitment maneuver, reducing the I:E ratio to 1:1 or initiate an inverse I:E ratio whereby the inspiratory time is longer than the expiratory time, increasing FiO2, or any combination thereof.

Generating an alert may comprise providing an audible alert such as a tone or pre-recorded message. The alert may comprise a visual indication on the display 16 and/or the pneumoperitoneum dashboard 18. It should be appreciated that various other types or embodiments of visual alerts may be envisioned within the scope of the present disclosure. For example, the alert may comprise both an audible and a visual component.

Providing a recommendation may comprise providing clinical decision support to guide the clinician in managing the patient ventilation. In one embodiment, a change in ventilation setting may be recommended. For example, it may be recommended to decrease tidal volume, increase respiratory rate, manage PIP to be less than 35 cm H2O, increase positive end-expiratory pressure (PEEP), perform a recruitment maneuver, reduce the I:E ratio to 1:1 or initiate an inverse I:E ratio whereby the inspiratory time is longer than the expiratory time, increase FiO2, or any combination thereof. In another embodiment, a change in a procedural parameter may be recommended. For example, it may be recommended to change the patient position, such as reducing the table tilt from a steep Trendelenburg to a mild Trendelenburg. It should be appreciated, however, that other recommendations may be envisioned within the scope of the present disclosure.

The method 300 may also comprise generating an indicator representative of the patient's response to the Trendelenburg position. In one embodiment, the indicator may be a numeric value. For example, the numeric value may be a number between 1 and 10, wherein 1 represents a patient responding positively to Trendelenburg and 10 represents a patient in distress. In another embodiment, the indicator is color. For example, colors along a green/yellow/red spectrum may be used to qualify a patient's response to Trendelenburg, where green represents a patient responding positively to Trendelenburg and red represents a patient in distress. In yet another embodiment, the indicator may be a combination of a numeric value and a color. It should be appreciated however, that other embodiments of the indicator may be envisioned.

The method 300 may also comprise displaying a pneumoperitoneum dashboard 18, as shown and described with respect to FIG. 2, for example. The dashboard 18 may present at least a physiological parameter 20, a ventilation parameter 22 and a procedural parameter 24, but may present a combination thereof. Additionally, the dashboard may present a plurality physiological parameters, a plurality of ventilation settings, a plurality of procedural parameters, or a combination thereof.

The pneumoperitoneum dashboard 18 may be further configured to present the indicator. As described above with reference to FIG. 2, the indicator is representative of the patient's response to the Trendelenburg position. In one embodiment, the indicator may be a numeric value. For example, the numeric value may be a number between 1 and 10, wherein 1 represents a patient responding positively to Trendelenburg and 10 represents a patient in distress. In another embodiment, the indicator is color. For example, colors along a green/yellow/red spectrum may be used to qualify a patient's response to Trendelenburg, where green represents a patient responding positively to Trendelenburg and red represents a patient in distress. In yet another embodiment, the indicator may be a combination of a numeric value and a color. It should be appreciated however, that other embodiments of the indicator may be envisioned.

The method 300 may also comprise insufflating the patient.

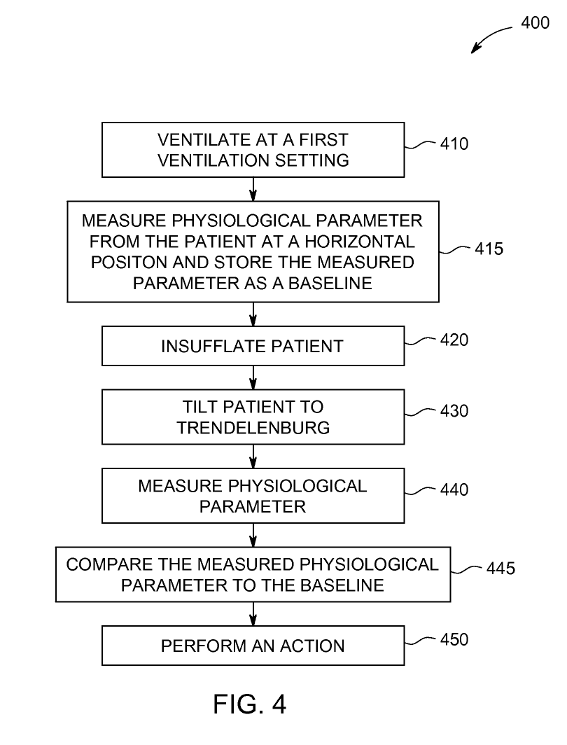

Referring to FIG. 4, a method 400 of ventilating a patient comprises ventilating the patient at a first ventilation setting at step 410.

At step 415, baseline values are measured and recorded for one or more physiological parameters from the patient. The patient physiological parameter may comprise IAP, PIP, ETCO2, SPO2, BP, APP, ICP, or any combination thereof. These baseline values are stored in the memory of the ventilator.

At step 430, the patient is tilted from a horizontal position to Trendelenburg position.

When the patient is in the Trendelenburg position, one or more patient physiological parameters are measured at step 440.

At step 445, the measured patient physiological parameter or parameters are compared to the baseline value measured and recorded before the patient was tilted to a more severe Trendelenburg position.

At step 450, an action is performed. The action is based on the comparison between the baseline values and the measured physiological parameter when the patient is in the second position. The action may be modifying the first ventilation setting to a second ventilation setting, generating an alert, providing a recommendation, or any combination thereof.

Modifying the first ventilation setting to a second ventilation setting may comprise modifying at least one of tidal volume, respiratory rate, inspired oxygen fraction, I:E ratio, PEEP, or a combination thereof. It may also comprise the processor 14 decreasing tidal volume, increasing respiratory rate, managing PIP to be less than 35 cm H2O, increasing PEEP, performing a recruitment maneuver, reducing the I:E ratio to 1:1 or initiate an inverse I:E ratio whereby the inspiratory time is longer than the expiratory time, increasing FiO2, or any combination thereof. For example, with respect to FIG. 2, the processor may be driving from a set RR of 10 to an adapted RR of 17. In another example, the processor may be driving from a set TV of 700 mL to an adapted TV of 420 mL.

Generating an alert may comprise providing an audible alert such as a tone or pre-recorded message. The alert may comprise a visual indication on the display 16 or the pneumoperitoneum dashboard 18. It should be appreciated that various other types or embodiments of visual alerts may be envisioned within the scope of the present disclosure. For example, the alert may comprise both an audible and a visual component.

Providing a recommendation may comprise providing clinical decision support to guide the clinician in managing the patient ventilation. In one embodiment, a change in ventilation setting may be recommended. For example, it may be recommended to decrease tidal volume, increase respiratory rate, manage PIP to be less than 35 cm H2O, increase positive end-expiratory pressure (PEEP), perform a recruitment maneuver, reduce the I:E ratio to 1:1 or initiate an inverse I:E ratio whereby the inspiratory time is longer than the expiratory time, increase FiO2, or any combination thereof. In another embodiment, a change in a procedural parameter may be recommended. For example, it may be recommended to change the patient position, such as reducing the table tilt from a Steep Trendelenburg to a mild Trendelenburg. It should be appreciated, however, that other recommendations may be envisioned within the scope of the present disclosure.

The method 400 may comprise insufflating the patient's abdomen at step 420. This step may be performed after ventilation begins at 410, or it may be performed prior to ventilation at step 410.

The method 400 may also comprise generating an indicator representative of the patient's response to the Trendelenburg position. In one embodiment, the indicator may be a numeric value. For example, the numeric value may be a number between 1 and 10, wherein 1 represents a patient responding positively to Trendelenburg and 10 represents a patient in distress. In another embodiment, the indicator is color. For example, colors along a green/yellow/red spectrum may be used to qualify a patient's response to Trendelenburg, where green represents a patient responding positively to Trendelenburg and red represents a patient in distress. In yet another embodiment, the indicator may be a combination of a numeric value and a color. It should be appreciated however, that other embodiments of the indicator may be envisioned.

The method 400 may also comprise displaying a pneumoperitoneum dashboard 18, as shown and described with respect to FIG. 2, for example. The dashboard 18 may present at least a physiological parameter 20, a ventilation parameter 22 and a procedural parameter 24, but may present a combination thereof. Additionally, the dashboard may present a plurality physiological parameters, a plurality of ventilation settings, a plurality of procedural parameters, or a combination thereof.

The pneumoperitoneum dashboard 18 may be further configured to present the indicator. As described above with reference to FIG. 2, the indicator is representative of the patient's response to the Trendelenburg position. In one embodiment, the indicator may be a numeric value. For example, the numeric value may be a number between 1 and 10, wherein 1 represents a patient responding positively to Trendelenburg and 10 represents a patient in distress. In another embodiment, the indicator is color. For example, colors along a green/yellow/red spectrum may be used to qualify a patient's response to Trendelenburg, where green represents a patient responding positively to Trendelenburg and red represents a patient in distress. In yet another embodiment, the indicator may be a combination of a numeric value and a color. It should be appreciated however, that other embodiments of the indicator may be envisioned.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

I claim:

1. A method of ventilating a patient, comprising:
measuring a patient physiological parameter while the patient is in a first position;
recording the measured patient physiological parameter as a baseline;
tilting the patient from the first position to a second position;
measuring the patient physiological parameter when the patient is in the second position; and
performing an action based on a change in the measured physiological parameter from the baseline when the patient is in the second position, wherein the action is at least one of modifying a ventilation parameter, generating an alert, and providing a recommendation.

2. The method of claim 1, wherein the physiological parameter is at least one of intra-abdominal pressure, peak inspiratory pressure, end-tidal CO2, blood oxygen saturation, blood pressure, airway plateau pressure, and intracranial pressure.

3. The method of claim 1, wherein the modifying a ventilation parameter includes modifying at least one of tidal volume, respiratory rate, inspired oxygen fraction, I:E ratio, and positive end expiratory pressure.

4. The method of claim 1, wherein the first position is the horizontal supine position.

5. The method of claim 1, wherein the second position is between 1 and 30 degrees from horizontal.

6. The method of claim 1, wherein the second position is between 30 and 45 degrees from horizontal.

7. The method of claim 1, further comprising:
displaying a pneumoperitoneum dashboard, the dashboard presenting the physiological parameter, a ventilation parameter and a procedural parameter.

8. The method of claim 1, wherein the procedural parameter is at least one of insufflation gas type, table angle, and operative time.

9. The method of claim 1, further comprising:
insufflating the patient.

10. The method of claim 1, further comprising:
generating an indicator representative of the patient's response to the second position.

11. The method of claim 1, wherein the recommendation comprises at least one of adjusting a ventilation setting and adjusting patient position.

12. A method of ventilating a patient, comprising:
ventilating the patient at a first ventilation setting;
measuring at least one physiological parameter from the patient at a horizontal position and storing the measured parameter as a baseline;
tilting the patient from the horizontal position to a Trendelenburg position;
measuring a patient physiological parameter when the patient is in the Trendelenburg position;
comparing the measured patient physiological parameter to the baseline; and
performing an action based on the comparison of the measured physiological parameter to the baseline, wherein the action is at least one of modifying the first ventilation setting to a second ventilation setting, generating an alert, and providing a recommendation.

13. The method of claim 12, further comprising:
insufflating the patient's abdomen.

14. The method of claim 12, wherein the physiological parameter is at least one of intra-abdominal pressure, peak inspiratory pressure, end-tidal CO2, blood oxygen saturation, blood pressure, airway plateau pressure, and intracranial pressure.

15. The method of claim 12, wherein the modifying the first ventilation setting to a second ventilation setting includes modifying at least one of tidal volume, respiratory rate, inspired oxygen fraction, I:E ratio, and positive end expiratory pressure.

16. The method of claim 12, wherein the recommendation comprises at least one of adjusting the ventilation setting and adjusting patient position.

17. The method of claim 12, wherein the alert is indicative of the measured physiological parameter value being outside a threshold value.

18. The method of claim 12, further comprising:
displaying a pneumoperitoneum dashboard, the dashboard presenting the physiological parameter, the ventilation setting and a procedural parameter.

19. The method of claim 18, wherein the procedural parameter is at least one of insufflation gas type, table angle, and operative time.

20. The method of claim 12, further comprising:
generating an indicator representative of the patient's response to the Trendelenburg position.

21. A system for monitoring a patient ventilated in the Trendelenburg position, comprising:
a processor configured to receive a physiological parameter, a ventilation parameter and a procedural parameter, the controller further configured to generate an indicator representative of the patient's response to the Trendelenburg position; and
a display operatively connected to the processor and configured to present a pneumoperitoneum dashboard comprising the physiological parameter, the ventilation parameter, the procedural parameter, and the indicator.
